(19) United States
(12) Patent Application Publication
(10) Pub. No.: US 2020/0297755 A1
(43) Pub. Date: Sep. 24, 2020

(54) PHARMACEUTICAL COMPOSITION FOR TREATING JOINT INFLAMMATION

(71) Applicant: JOINTHERAPEUTICS S.R.L., Como (IT)

(72) Inventors: Giulio Bianchini, Como (IT); Lanfranco Callegaro, Como (IT)

(73) Assignee: JOINTHERAPEUTICS S.R.L., Como (IT)

(21) Appl. No.: 16/652,211

(22) PCT Filed: Oct. 3, 2018
(86) PCT No.: PCT/IB2018/057686

§ 371 Date: Mar. 30, 2020

(30) Foreign Application Priority Data
Oct. 5, 2017 (IT) 102017000111939

(51) Int. Cl. A61K 31/728; A61K 9/00; A61K 47/26; A61K 47/34; A61K 47/42; A61K 47/36; A61K 47/02; A61P 19/02

(52) U.S. Cl. CPC A61K 31/728; A61K 9/0019; A61K 47/26; A61P 19/02; A61K 47/42; A61K 47/36; A61K 47/02; A61K 47/34

(57) ABSTRACT

The present invention discloses a pharmaceutical composition comprising at least one bioactive substance and at least one neutral salt consisting of a polyamino-saccharide cation and an anion, for use in the treatment of articular tissues affected by arthropathies, wherein said pharmaceutical composition reduces expression of the key receptors involved in the genesis the inflammatory cascade and efficaciously maintains performance for a prolonged period of time.

### FIELD OF THE INVENTION

The present invention relates to a pharmaceutical composition comprising at least one bioactive substance and at least one neutral salt consisting of a polyamino-saccharide cation and an anion, for use in the treatment of articular tissues affected by arthropathies, wherein said pharmaceutical composition reduces expression of receptor GLT3 and of enzyme MMP13, and increases production of collagen II.

### BACKGROUND ART

Galectins are a family of proteins which are defined by their binding specificity for β-galactoside sugars, such as N-acetyl-lactosamine, which can be bound to proteins via N-glycosylation or O-glycosylation. There are 15 galectins known in mammals, which are encoded by LGALS genes and are numbered consecutively, but only −1, −2, −3, −4, −7, −8, −9, −10, −12, and −13 have been identified in humans.

These are located in intracellular or extracellular locations. In the latter case, they perform bivalent or multivalent interactions with glycans on cell surfaces and induce various cellular responses, including the production of cytokines and other inflammatory mediators, cell adhesion, migration, and apoptosis. Furthermore, they can form lattices with membrane glycoprotein receptors and modulate the properties of the receptors. Intracellular galectins can participate in signalling pathways and alter biological responses, including apoptosis, cell differentiation, and cell motility. Current evidence indicates that galectins play an important role in acute and chronic inflammatory responses, as well as in other different pathological processes.

Metalloproteases (or metalloproteinases) constitute a family of protease group enzymes, which are classified according to the nature of the most important functional groups in the active site thereof. Matrix metalloproteinases (or MMPs) are enzymes which need zinc ions as a cofactor and can alter the properties of the basal lamina. The 21 members of MMP are the most potent degradative enzymes in the extracellular matrix and are distinguished by the structural component of the matrix on which they act: e.g. collagenases (MMP-1, MMP-8, MMP-13) act on collagen. They are normally produced by the majority of the cells in the organism and are involved in a large number of tissue remodelling processes, including those of a physiological nature, associated with growth, development and shelter, and those of a pathological nature, such as degenerative and inflammatory diseases. Metalloproteinases are hyperexpressed in many inflammatory pathologies, therefore the inhibition of metalloproteinase activity can consequently determine a marked reduction in the inflammatory cascade.

An object of the present invention is therefore to provide a product which reduces the expression of these receptors and enzymes, so as to act therapeutically on inflammatory pathologies, while also offering a high acceptability profile thereof from a medical and a pharmaceutical viewpoint.

### SUMMARY OF THE INVENTION

Said object is achieved by a pharmaceutical composition comprising at least one bioactive substance and at least one neutral salt consisting of a polyamino-saccharide cation and an anion, as stated in claim 1, for use in the treatment of articular tissues affected by arthropathies, wherein said pharmaceutical composition reduces expression of receptor GLT3 and of enzyme MMP13, and increases production of collagen II.

For the purposes of the present invention, the term “arthropathy” means any joint disease selected from: degenerative arthropathy, otherwise known as arthritis, traumatic arthropathy, ankylosing spondylitis, Reiter's syndrome, juvenile rheumatoid arthritis, neuropathic arthropathy, dysmetabolic arthropathy, such as gout, ochronosis and alkaptonuria, para-articular rheumatic disorder, such as canalicular syndrome, myositis, algodystrophy, and tenosynovitis.

### BRIEF DESCRIPTION OF THE FIGURES

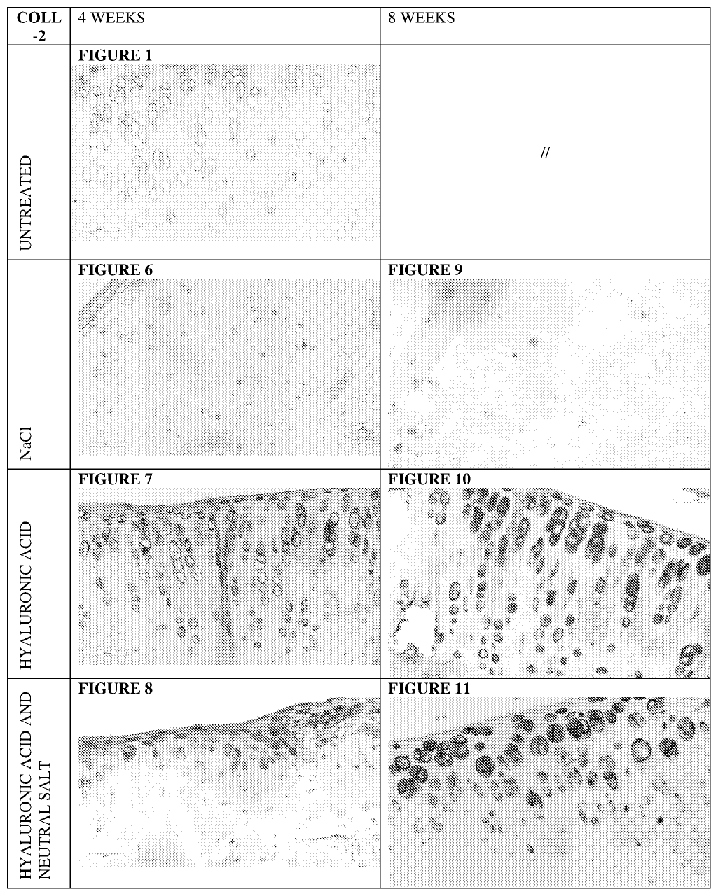

The characteristics and advantages of the present invention will become apparent from the following detailed description, the embodiments provided by way of non-limiting examples and the figures annexed hereto, wherein FIGS. 1-35 show images (40× magnification) of histological specimens coloured with specific probes for the different markers studied, as described in Example 22.

### DETAILED DESCRIPTION OF THE INVENTION

The invention relates, therefore, to a pharmaceutical composition for use in the treatment of joint tissues affected by arthropathy, wherein said pharmaceutical composition reduces expression of receptor GLT3 and enzyme MMP13 and increases production of collagen II, said pharmaceutical composition comprising:
at least one bioactive substance selected from collagen, fibrinogen, fibrin, alginic acid, sodium alginate, potassium alginate, magnesium alginate, hyaluronic acid, sodium hyaluronate, potassium hyaluronate, iron hyaluronate, calcium hyaluronate, magnesium hyaluronate, zinc hyaluronate, hyaluronic acid derivate, cellulose, chondroitin sulfate, dermatan sulfate, keratan sulfate, heparin, heparan sulfate, laminin, fibronectin, elastin, polylactic acid, polyglycolic acid, poly(lactic-co-glycolic acid), polycaprolactone, gelatin, albumin, poly(glycolide-co-caprolactone), poly(glycolide-co-trimethylene carbonate), hydroxyapatite, tricalcium phosphate, dicalcium phosphate, demineralized bone matrix, and mixtures thereof, and
at least one neutral salt consisting of a polyamino-saccharide cation and an anion, wherein the polyamino-saccharide cation consists of the following three repeating units:

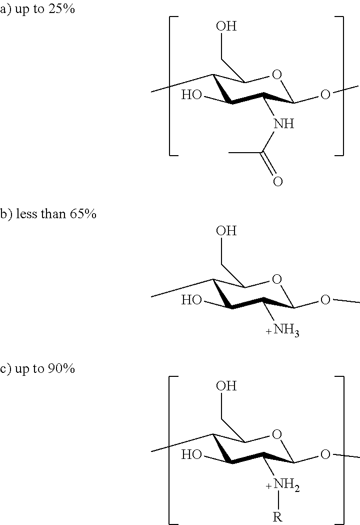

wherein R is an aldose or ketose moiety,
and wherein the anion is monovalent, bivalent, or trivalent.

The definition of “neutral salt” includes all the polymorphic forms (both amorphous and crystalline) and the co-crystalline forms, as well as the anhydrous, hydrated, and solvate forms.

The repeating units b) and c) are shown having the positive charge on the nitrogen atom, however, other forms of conjugated acid, in equilibrium with the most likely ammonium form shown, cannot be excluded.

For the purposes of the present invention “hyaluronic acid derivate” means:
hyaluronic acid ester, wherein a part or all of the carboxylic acid groups are esterified with aliphatic, aromatic, arylaliphatic, cycloaliphatic, or heterocyclic series alcohols, as described, for example, in EP0216453,
self-crosslinked hyaluronic acid ester, wherein a part or all of the carboxylic acid groups are esterified with alcoholic groups from the same polysaccharide chain or other chains, as described, for example, in EP0341745,
a crosslinked hyaluronic acid ester compound, wherein a part or all of the carboxylic acid groups are esterified with aliphatic, aromatic, arylaliphatic, cycloaliphatic, or heterocyclic series polyalcohols, generating cross-linking by means of spacer groups, as described, for example, in EP0265116,
succinic acid hemiester or succinic acid ester heavy metal salt with hyaluronic acid or with partial or total hyaluronic acid esters, as described, for example, in WO96/357207,
O-sulfated derivative, as described, for example, in WO95/25751, or N-sulfated derivative, as described, for example, in PCT/EP98/01973,
amide of hyaluronic acid or of the derivatives thereof listed above, as described, for example, in EP1095064,
   or a mixture thereof.

In preferred embodiments, said pharmaceutical composition comprises one bioactive substance, as defined above, and one neutral salt, as defined above.

It has surprisingly been found that the composition envisaged in the invention is capable of reducing expression of receptor GLT3 and of enzyme MMP13, i.e. expression of the key receptors involved in the genesis of the inflammatory cascade, while also advantageously increasing production of collagen II at the same time.

In certain preferred embodiments, the pharmaceutical composition envisaged in the invention also further reduces expression of receptor GLT1.

In other preferred embodiments, the pharmaceutical composition envisaged in the invention also further reduces expression of enzyme MMP3.

In further preferred embodiments, the pharmaceutical composition envisaged in the invention also further reduces expression of receptor GLT1 and enzyme MMP3. Advantageously, the composition envisaged in the invention reduces expression of further key receptors involved in the genesis of the inflammatory cascade.

With reference to the neutral salt, preferably, R is a moiety of formula (1):

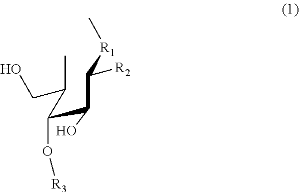

wherein R1 is —CH₂— or —CO—,
R₂ is —OH, or —NHCOCH₃,
R₃ is H, monosaccharide, disaccharide, or oligosaccharide,
   or R is a moiety of formula (2):

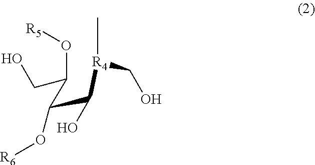

R₄ is —CH—,
R₅ and R₆ are, independently of each other, H, monosaccharide, disaccharide, or oligosaccharide.

Preferably, R₃, R₅ e R₆ are, independently of one another, H, glucose, galactose, arabinose, xylose, mannose, lactose, trehalose, gentiobiose, cellobiose, cellotriose, maltose, maltotriose, chitobiose, chitotriose, mannobiose, melibiose, fructose, N-acetyl glucosamine, N-acetylgalactosamine, or a combination thereof.

More preferably, R₃ is H, glucose, galactose, mannose, N-acetylglucosamine, N-acetylgalactosamine, or a combination thereof.

In particularly preferred embodiments, R is a moiety of lactose or galactose.

Preferably, in the polyamino-saccharide cation, the repeating unit a) is present in a percentage of 5% to 20%.

More preferably, in the polyamino-saccharide cation, the repeating unit a) is present in a percentage of 7% to 18%.

Preferably, in the polyamino-saccharide cation, the repeating unit b) is present in a percentage of 5% to 45%.

More preferably, in the polyamino-saccharide cation, the repeating unit b) is present in a percentage of 20% to 40%.

Preferably, in the polyamino-saccharide cation, the repeating unit c) is present in a percentage of 45% to 90%.

More preferably, in the polyamino-saccharide cation, the repeating unit c) is present in a percentage of 50% to 70%.

In preferred embodiments, the polyamino-saccharide cation consists of:

5% to 20% repeating unit a), 5% to 45% repeating unit b), and 45% to 90% repeating unit c).

In more preferred embodiments, the polyamino-saccharide cation consists of:

7% to 18% repeating unit a), 20% to 40% repeating unit b), and 50% to 70% repeating unit c).

Preferably, the anion is chloride, bromide, fluoride, iodide, acetate, trifluoroacetate, carbonate, bicarbonate, sulfate, bisulfate, C1-C10 alkylsulfate, C1-C6 alkylsulfonate, C6-C10 arylsulfonate, nitrate, hydrogen phosphate, dihydrogen phosphate, orthophosphate, oxalate, fumarate, ascorbate, citrate, gluconate, lactate, formate, tartrate, succinate, mandelate, p-toluenesulfonate, carboxylate, saccharate, benzoate, or a mixture thereof.

More preferably, the anion is chloride, bromide, acetate, sulfate, trifluoroacetate, methanesulfonate, orthophosphate or, nitrate, or a mixture thereof.

Preferably, the weight average molecular weight (Mw) of the neutral salt of the invention is up to 2500 kDa, more preferably up to 250 kDa-1500, and even more preferably up to 400 kDa-900 kDa.

Preferably, the number average molecular weight (Mn) of the neutral salt of the invention is up to 2000 kDa, more preferably up to 100 kDa-1000, and even more preferably up to 200 kDa-500 kDa.

Preferably, said neutral salt and said bioactive substance are in a weight ratio of 10:1 to 1:50.

Preferably, the pharmaceutical composition according to the invention comprises up to 10 wt % of said neutral salt, based on the weight of the pharmaceutical composition, and more preferably, up to 5 wt % of said neutral salt. Particularly preferable are pharmaceutical compositions wherein the amount of said neutral salt is 0.5-5 wt %, based on the weight of the composition.

In first preferred embodiments of the pharmaceutical composition, said bioactive substance is selected from hyaluronic acid, sodium hyaluronate, potassium hyaluronate, iron hyaluronate, calcium hyaluronate, magnesium hyaluronate, zinc hyaluronate, hyaluronic acid derivate, and mixtures thereof. In these embodiments, the amount of said neutral salt is preferably greater than or equal to that of said bioactive substance. In these embodiments, preferably, said neutral salt and said bioactive substance are in a weight ratio of 5:1 to 1:5, preferably 4:1 to 1:4, and more preferably 3:1 to 1:3.

In preferred second embodiments of the pharmaceutical composition, said bioactive substance is selected from hydroxyapatite, tricalcium phosphate, dicalcium phosphate, demineralized bone matrix, and mixtures thereof. In these embodiments, the amount of said neutral salt is preferably less than or equal to that of said bioactive substance. In these embodiments, preferably, said neutral salt and said bioactive substance are in a weight ratio of 1:10 to 1:45, preferably 1:20 to 1:40, and more preferably 1:25 to 1:35. Said second embodiments are further preferred when the active substance is selected from hydroxyapatite and tricalcium phosphate and mixtures thereof.

In particularly preferred embodiments, the present invention relates to a pharmaceutical composition comprising at least one neutral salt consisting of a polyamino-saccharide cation and an anion, as described above, and hydroxyapatite.

The pharmaceutical composition may be administered orally, intramuscularly, intravenously, intra-articularly, transdermally, subdermally, or topically externally or internally, for example by surgical means.

Preferably, said pharmaceutical composition has a pH of 6-8.

Preferably, said pharmaceutical composition has an ionic strength of 50-150 mM.

Preferably, said pharmaceutical composition is in the form of an aqueous solution for injection.

Preferably, said pharmaceutical composition further comprises a buffer, more preferably a buffer selected from: saline phosphate buffer, ammonium acetate buffer, arginine buffer, glycine buffer, meglumine buffer, Glucono Delta Lactone buffer, tromethamine buffer and mixtures thereof.

In preferred embodiments, said buffer is a saline phosphate buffer.

More preferably, said pharmaceutical composition in the form of an aqueous solution for injection further comprises a buffer. In preferred embodiments, said buffer is a saline phosphate buffer.

The pharmaceutical composition may further comprise pharmaceutically acceptable excipients.

Suitable pharmaceutically acceptable excipients include, for example, isotonic regulators, solvents, stabilisers, chelating agents, diluents, binders, disintegrators, lubricants, glidants, colorants, suspending agents, surfactants, cryoprotectants, preservatives, and antioxidants.

For the purposes of the present invention, said neutral salt may be prepared by a process comprising the following steps:

i) providing a polyamino-saccharide polymer consisting of repeating units a) and b),
ii) reacting said polyamino-saccharide polymer with a monosaccharide, disaccharide, or oligosaccharide, in aqueous solution,
iii) adding an amino-borane,
iv) adding an acid to a pH value lower than 4,
v) adding an organic solvent, thus precipitating the neutral salt, and
vi) separating the precipitated neutral salt.

It has surprisingly been observed that the amino-boranes present a marked selectivity in the reduction of the imino group compared with the carbonyl group and are compatible with the aqueous environment; at the same time, the formation of a salt owing to the reaction with an acid reduces the time needed for purification of the end product and to neutralise the excess hydride ions, thereby advantageously avoiding the use of bacteriostats. Therefore, the process as a whole offers the advantage of improved acceptability from a medical and pharmaceutical point of view, since the purity of the end product has been significantly increased, as well as the overall rapidity of the preparation.

Preferably, said polyamino-saccharide polymer consists of 5% to 95% repeating unit a) and 95% to 5% repeating unit b).

Said monosaccharide, disaccharide, or oligosaccharide corresponds to the definition given above for the moiety R.

Preferably, the aqueous solution in step ii) is an aqueous solution of acid acetic with a concentration by weight of 0.5-5%, and more preferably of 0.5-2.5%.

Said amino-borane is preferably 2-methylpyridine borane, 5-ethyl-2-methylpyridine borane, pyridine borane, trimethylamine borane, triethylamine borane, dimethylamine borane, tert-butylamine borane, or a mixture thereof. More preferably, said amino-borane is 2-methylpyridine borane, 5-ethyl-2-methylpyridine borane, or a mixture thereof.

The amino-boranes may be used as such or may be previously solubilised or dispersed in water-miscible organic solvents such as alcohols. The most preferred among said alcohols are methanol, ethanol, 2-propanol, or a mixture thereof.

The term “acid” means the corresponding acid of the anion described above.

The term “organic solvent” means an organic water-miscible solvent capable of lowering the dielectric constant of the aqueous reaction solution. Suitable organic solvents are acetone, methanol, ethanol, 2-propanol, or a mixture thereof, and preferably the organic solvent is 2-propanol.

Optionally, the precipitate separated in step vi) is washed with mixtures of water and organic solvent, with water in percentages of up to 60%, and more preferably up to 40%.

Preferably, the molar ratio of monosaccharide, disaccharide, or oligosaccharide and the repeating unit b) of the polyamino-saccharide polymer is of 0.5 to 30, more preferably 1 to 20, and even more preferably 1 to 5.

Preferably, the molar ratio of amino-boran and the repeating unit b) of the polyamino-saccharide polymer is of 0.75 to 20, more preferably 1 to 10, and even more preferably 1 to 3.

It should also be understood that all aspects identified as favourable and advantageous for the neutral salt should be deemed equally preferable and advantageous also for the preparation process, the compositions, the biomaterials, and the uses described above. It should furthermore be understood that all the possible combinations of the preferred aspects of the neutral salt, the preparation process, the compositions, the biomaterials, and the uses stated above are likewise preferred.

Below are working examples of the present invention provided for illustrative purposes.

### EXAMPLES

### General Procedure for the Preparation Process:

A monosaccharide, disaccharide, or oligosaccharide (0.30-0.20 M), water, acetic acid (0.10-0.20 M) and chitosan having 5% to 20% repeating units a) (0.10 M) were loaded into a reactor. The mixture thus obtained was heated to 60° C. for 2 hours. Then, under the same conditions, an amino-borane (0.10-0.25 M) was added gradually, after being dispersed in an alcohol (10-20%), and the system was left under stirring at 60° C. for 2 hours. Subsequently, an aqueous solution of acid (2-4 N) was added drop by drop until a pH value of approximately 2 was reached. Next, the system was cooled to room temperature and the product was precipitated by adding an organic solvent; the precipitate was decanted, the supernatant removed, and the solid washed a first time with a (30:70) water:organic solvent mixture, and then several times with a (15:85) water:organic solvent mixtures, and a final time with organic solvent. Finally, the solid thus obtained was dried under reduced pressure and controlled temperature conditions.

### Example 1

Lactose (36 g), water (400 mL), acetic acid (100%), and chitosan (12 g) were loaded into a reactor and the mixture thus obtained heated to 60° C. for 2 hours. Next, under the same conditions, 5-ethyl-2-methylpyridine borane (8 g) previously dispersed in methanol (50 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 2

Lactose (22 g), water (400 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (50 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 3

Lactose (36 g), water (400 mL), acetic acid (100%), and chitosan (12 g) were loaded into a reactor and the mixture thus obtained heated to 60° C. for 2 hours. Next, under the same conditions, 2-methylpyridine borane (14 g) previously dispersed in methanol (80 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 4

Lactose (36 g), water (500 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (80 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 5

Lactose (36 g), water (500 mL), acetic acid (100%), and chitosan (12 g) were loaded into a reactor and the mixture thus obtained heated to 60° C. for 2 hours. Next, under the same conditions, 5-ethyl-2-methylpyridine borane (8 g) previously dispersed in methanol (80 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding acetone. Subsequently, the precipitate was decanted, the supernatant removed, and the solid washed a first time with a (20:80) water:methanol mixture, and then several times with a (10:90) water:methanol mixtures, and a final time with methanol. Finally, the solid thus obtained was dried under reduced pressure and controlled temperature conditions.

### Example 6

Lactose (36 g), water (500 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (80 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding acetone. Subsequently, the precipitate was decanted, the supernatant removed, and the solid washed a first time with a (25:75) water:ethanol mixture, and then several times with a (15:85) water:ethanol mixtures, and a final time with ethanol. Finally, the solid thus obtained was dried under reduced pressure and controlled temperature conditions.

### Example 7

Lactose (36 g), water (400 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (50 mL) was gradually added and the system was left under stirring at 60° C. for 2 hours. Subsequently, an aqueous solution of sulfuric acid (2 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 8

Lactose (36 g), water (400 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (50 mL) was gradually added and the system was left under stirring at 60° C. for 2 hours. Subsequently, an aqueous solution of orthophosphoric acid (2 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 9

Lactose (36 g), water (400 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (50 mL) was gradually added and the system was left under stirring at 60° C. for 2 hours. Subsequently, an aqueous solution of trifluoroacetic acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 10

Lactose (36 g), water (400 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (50 mL) was gradually added and the system was left under stirring at 60° C. for 2 hours. Subsequently, an aqueous solution of methanesulfonic acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 11

Lactose (36 g), water (400 mL), acetic acid (100%), and chitosan (12 g) were loaded into a reactor and the mixture thus obtained heated to 60° C. for 2 hours. Next, under the same conditions, 5-ethyl-2-methylpyridine borane (10 g) previously dispersed in methanol (50 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Example 12

Galactose (20 g), water (400 mL), acetic acid (100%), and chitosan (12 g) were loaded into a reactor and the mixture thus obtained heated to 60° C. for 2 hours. Next, under the same conditions, 2-methylpyridine borane (8 g) previously dispersed in methanol (50 mL) was gradually added and the system was left under stirring at 60° C. for 2 hours. Subsequently, an aqueous solution of hydrochloric acid (4 N) was added drop by drop until a pH value of approximately 2 was reached. Next, the system was cooled to room temperature and the product was precipitated by adding 2-propanol; Subsequently, the precipitate was decanted, the supernatant removed, and the solid washed a first time with a (30:70) water:2-propanol mixture, and then several times with a (15:85) water:2-propanol mixtures, and a final time with 2-propanol. Finally, the solid thus obtained was dried under reduced pressure and controlled temperature conditions.

### Results

Table 1 summarises the chemical and physical characteristics of the salts obtained in Examples 1-12 set out above. The polysaccharide derivatives were obtained with advantageous yields, easy modification of the degree of substitution, and a high degree of purity.

The corresponding percentages, in the three different repeating units, of the polyamino-saccharide cation was determined by ¹H-NMR analysis, as reported in the literature (N. D'Amelio et al. J. Phys. Chem. B 2013, 117, 13578).

TABLE 1

| | | | | Boron | Ammine |
|---|---|---|---|---|---|
| | Yield | | | residual | residual |
| EXAMPLES | (g) | CATION | ANION | (ppm) | (ppm) |
| 1 | 27 | a = 8%; | [Cl]⁻ | <50 | <50 |
| | | b = 32%; | | | |
| | | c = 60% | | | |
| 2 | 19 | a = 10%; | [Cl]⁻ | <50 | <50 |
| | | b = 63%; | | | |
| | | c = 27% | | | |
| 3 | 32 | a = 9%; | [Cl]⁻ | <50 | <50 |
| | | b = 9%; | | | |
| | | c = 82% | | | |
| 4 | 27 | a = 8%; | [Cl]⁻ | <50 | <50 |
| | | b = 37%; | | | |
| | | c = 55% | | | |
| 5 | 28 | a = 9%; | [Cl]⁻ | <50 | <50 |
| | | b = 30%; | | | |
| | | c = 61% | | | |
| 6 | 26 | a = 15%; | [Cl]⁻ | <50 | <50 |
| | | b = 27%; | | | |
| | | c = 56% | | | |
| 7 | 28 | a = 12%; | [SO4]²⁻ | <50 | <50 |
| | | b = 29%; | | | |
| | | c = 59% | | | |
| 8 | 31 | a = 10%; | [H₂PO₄]⁻ | <50 | <50 |
| | | b = 30%; | | | |
| | | c = 60% | | | |
| 9 | 32 | a = 8%; | [CF₃COO]⁻ | <50 | <50 |
| | | b = 35%; | | | |
| | | c = 57% | | | |
| 10 | 31 | a = 17%; | [CH₃SO₃]⁻ | <50 | <50 |
| | | b = 21%; | | | |
| | | c = 62% | | | |
| 11 | 27 | a = 15%; | [Cl]⁻ | <50 | <50 |
| | | b = 22%; | | | |
| | | c = 63% | | | |
| 12 | 23 | a = 11%; | [Cl]⁻ | <50 | <50 |
| | | b = 0%; | | | |
| | | c = 89% | | | |

### Example 13

Composition with Polylactic Acid (Neutral Salt 1.00%, Polylactic Acid 0.50%)

The neutral salt obtained in Example 4 (0.630 g) was dissolved in water (25 ml) and the resulting solution mixed at room temperature for 1 hour. Subsequently, a sodium hydroxide solution (2.4 mL, 0.5 N) was added drop by drop under the same conditions and the resulting solution mixed for further 30 minutes. Next, the following were added in order, under the same conditions: a 10× solution of PBS (6.30 ml, PBS 10×: Na₂HPO₄ 81 Mm, NaH₂PO₄ 17.6 Mm, NaCl 1370 Mm, KCl 27 Mm), water (20 ml), polylactic acid (0.315 g) and water (9.30 ml). The mixture thus obtained was stirred at room temperature until a homogeneous system was obtained.

### Example 14

Composition with Collagen (Neutral Salt 1.00%, Collagen 1.00%)

The neutral salt obtained in Example 4 (0.630 g) was dissolved in water (25 ml) and the resulting solution mixed at room temperature for 1 hour. Subsequently, a sodium hydroxide solution (2.4 mL, 0.5 N) was added drop by drop under the same conditions and the resulting solution mixed for further 30 minutes. Next, the following were added in order, under the same conditions: a 10× solution of PBS (6.30 ml, PBS 10×: Na₂HPO₄ 81 Mm, NaH₂PO₄ 17.6 Mm, NaCl 1370 Mm, KCl 27 Mm), water (20 mL), polylactic acid (0.630 g) and water (9.30 mL). The mixture thus obtained was stirred at room temperature until a homogeneous system was obtained.

### Example 15

Composition with Chondroitin Sulfate (Neutral Salt 1.20%, Chondroitin Sulfate 0.40%)

The neutral salt obtained in Example 4 (0.756 g) was dissolved in water (25 ml) and the resulting solution mixed at room temperature for 1 hour. Subsequently, a sodium hydroxide solution (2.88 mL, 0.5 N) was added drop by drop under the same conditions and the resulting solution mixed for further 30 minutes. Next, the following were added in order, under the same conditions: a 10× solution of PBS (6.30 ml, PBS 10×: Na₂HPO₄ 81 Mm, NaH₂PO₄ 17.6 Mm, NaCl 1370 Mm, KCl 27 Mm), water (20 mL), chondroitin sulfate (0.252 g) and water (8.82 mL). The mixture thus obtained was stirred at room temperature until the chondroitin sulfate was completely dissolved.

### Example 16

Composition with Poly(Lactic-Co-Glycolic) Acid or PLGA (Neutral Salt 1.80%, PGLA 1.00%)

The neutral salt obtained in Example 4 (1.134 g) was dissolved in water (25 ml) and the resulting solution mixed at room temperature for 1 hour. Subsequently, a sodium hydroxide solution (4.32 mL, 0.5 N) was added drop by drop under the same conditions and the resulting solution mixed for further 30 minutes. Next, the following were added in order, under the same conditions: a 10× solution of PBS (6.30 ml, PBS 10×: Na₂HPO₄ 81 Mm, NaH₂PO₄ 17.6 Mm, NaCl 1370 Mm, KCl 27 Mm), water (20 mL), PLGA (0.630 g) and water (7.38 mL). The mixture thus obtained was stirred at room temperature until a homogeneous system was obtained.

### Example 17

Composition with Elastin (Neutral Salt 0.75%, Elastin 0.25%)

The neutral salt obtained in Example 4 (0.473 g) was dissolved in water (25 ml) and the resulting solution mixed at room temperature for 1 hour. Subsequently, a sodium hydroxide solution (1.80 mL, 0.5 N) was added drop by drop under the same conditions and the resulting solution mixed for further 30 minutes. Next, the following were added in order, under the same conditions: a 10× solution of PBS (6.30 ml, PBS 10×: Na₂HPO₄ 81 Mm, NaH₂PO₄ 17.6 Mm, NaCl 1370 Mm, KCl 27 Mm), water (20 mL), elastin (0.158 g) and water (11.10 mL). The mixture thus obtained was stirred at room temperature until a homogeneous system was obtained.

### Example 18

Composition with Potassium Alginate (Neutral Salt 1.00%, Potassium Alginate 0.75%)

The neutral salt obtained in Example 4 (0.630 g) was dissolved in water (25 ml) and the resulting solution mixed at room temperature for 1 hour. Subsequently, a sodium hydroxide solution (2.40 mL, 0.5 N) was added drop by drop under the same conditions and the resulting solution mixed for further 30 minutes. Next, the following were added in order, under the same conditions: a 10× solution of PBS (6.30 ml, PBS 10×: Na₂HPO₄ 81 Mm, NaH₂PO₄ 17.6 Mm, NaCl 1370 Mm, KCl 27 Mm), water (20 mL), potassium alginate (0.473 g) and water (9.30 mL). The mixture thus obtained was stirred at room temperature until the potassium alginate completely dissolved.

### Example 19

Composition with hydroxyapatite in tricalcium phosphate (neutral salt 1.94%, Hydroxyapatite 1.78%, Tricalcium Phosphate 57.58%)

The neutral salt obtained in Example 4 (0.163 g) was placed in water (2.64 ml) and mixed at room temperature for 1 hour and at 60° C. for 2 hours; subsequently, a sodium hydroxide solution (0.62 mL, 0.5 N) was added drop by drop under the same conditions and the resulting solution mixed for further 30 minutes. The solution thus obtained was then transferred, at room temperature, to a beaker containing hydroxyapatite (0.150 g) homogeneously dispersed in tricalcium phosphate (4.850 g). The liquid phase and the solid phase were then intimately mixed until a cement paste was obtained.

### Example 20

Composition with Tricalcium Phosphate in Hydroxyapatite (Neutral Salt 1.94%, Tricalcium Phosphate 1.78%, Hydroxyapatite 57.58%)

The neutral salt obtained in Example 4 (0.163 g) was placed in water (2.64 ml) and mixed at room temperature for 1 hour and at 60° C. for 2 hours; subsequently, a sodium hydroxide solution (0.62 mL, 0.5 N) was added drop by drop under the same conditions and the resulting solution mixed for further 30 minutes. The solution thus obtained was then transferred, at room temperature, to a beaker containing tricalcium phosphate (0.150 g) homogeneously dispersed in hydroxyapatite (4.850 g). The liquid phase and the solid phase were then intimately mixed until a cement paste was obtained.

### Example 21

Composition with Hyaluronic Acid (Neutral Salt 0.75%, Hyaluronic Acid 1.25%)

The neutral salt obtained in Example 4 (0.473 g) was dissolved in water (25 ml) and the resulting solution mixed at room temperature for 1 hour. Subsequently, a sodium hydroxide solution (1.8 mL, 0.5 N) was added drop by drop under the same conditions and the resulting solution mixed for further 30 minutes. Next, the following were added in order, under the same conditions: a 10× solution of PBS (6.30 ml, PBS 10×: Na₂HPO₄ 81 Mm, NaH₂PO₄ 17.6 Mm, NaCl 1370 Mm, KCl 27 Mm), water (20 mL), sodium hyaluronate (0.788 g) and water (11.10 mL). The mixture thus obtained was stirred at room temperature until a homogeneous system was obtained.

### Example 22

The following compositions to be tested were prepared:
0.9% NaCl saline solution
composition comprising 1.25% hyaluronic acid (shortly “HA”) in PBS 1× (PBS 1×: Na₂HPO₄ 8.1 mM, NaH₂PO₄ 1.76 mM, NaCl 137.0 mM, KCl 2.7 mM)
composition in Example 21

### Methods

Two slices were provided for each specimen (whole joint of rats-model: in vivo DMM, i.e. surgical destabilization of the medial meniscus).

For each slice, the paraffin was removed in solutions of ethanol in decreasing degrees, the slice was washed with PBS (phosphate buffered solution) for 10 minutes and immunostaining was performed for Collagen II, Metal Matrix Protein-3 (MMP-3), Metal Matrix Protein-13 (MMP-13), Galectin 1 (GLT-1) and Galectine 3 (GLT-3).

In short, after fixation, the slices were washed thoroughly with PBS and permeabilised by incubation in 0.3% hydrogen peroxide in PBS solution for 15 minutes. The slices were pre-treated in 0.2% Pronase PBS solution (Sigma-Aldrich, Saint Louis, US-MO) for 30 minutes at 37° C. for antigen unmasking. After washing, the slices were incubated at room temperature for one hour with Blocking Serum (Vectastain Universal Quick Kit, Vectors Laboratories, Burlingame, US-CA) to prevent non-specific bonds, then incubated with specific polyclonal rabbit antibodies against collagen II, MMP-3 (Assay Biotechnology Company, Inc., Sunnyvale, Calif.), MMP-13 (BioVision, Inc. Headquarters, California, US), Galectin 1 (NSJ Bioreagents, San Diego, Calif.) Galectin 3 (Assay Biotechnology Company, Inc., Sunnyvale, Calif.) overnight at 4° C.

After rinsing in PBS, the slices were incubated with anti-rabbit HRP-conjugated secondary antibody (Bethyl Laboratories, Montgomery, US-TX) and a streptavidin-peroxidase complex (Vectastain Universal Quick Kit). Finally, reactions were developed using a Vector® NovaRED™ Substrate Kit for peroxidase (Vectors Laboratories, Burlingame, US-CA). Negative controls were included, omitting the primary antibody, to verify specificity and performance of the reagents applied. Finally, immunohistochemical staining was assessed qualitatively on the surface of the specimens.

The images shown in the Figures were acquired using a digital pathology slide scanner (Aperio Digital Pathology Slide Scanners, Leica Biosystems) at 40× magnification.

### Results

FIGS. 1-5 refer to untreated specimens, which are therefore comparative specimens: FIG. 1. Collagen II expression on specimen of joint of rat subjected to destabilisation of medial meniscus. Presence of Collagen II expressed according to colour intensity (greyscale).

As expected, specimens obtained from untreated animals showed a low presence of Collagen II in the cartilaginous tissue, thereby confirming effectiveness of the chosen experimental model and the pathological course.

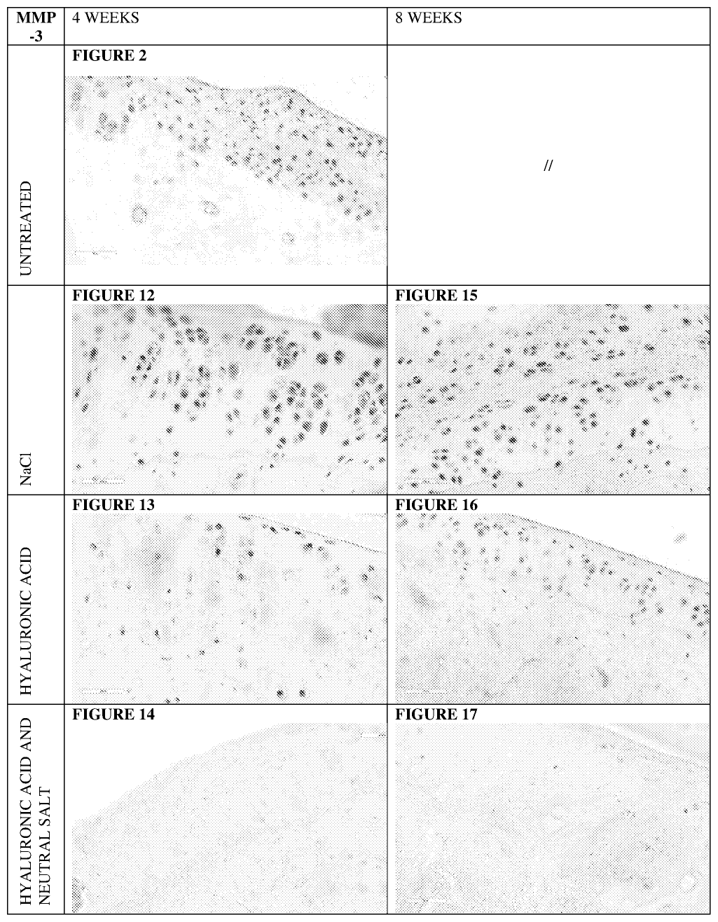

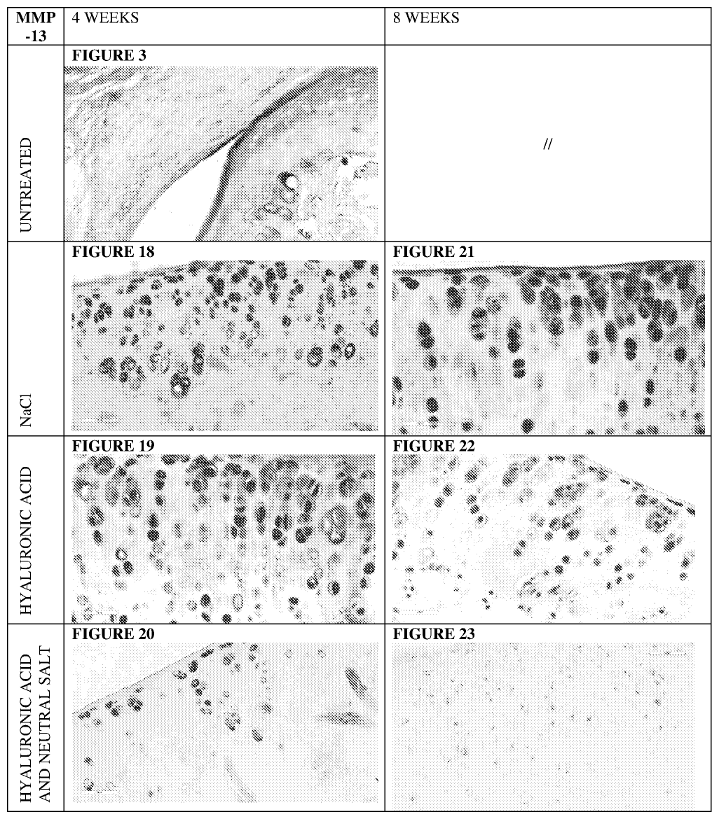

FIG. 2. MMP-3 expression on specimen of joint of rat subjected to destabilisation of medial meniscus. Presence of MMP-3 expressed according to colour intensity (greyscale).

As expected, specimens obtained from untreated animals showed a marked presence of MMP-3 in the cartilaginous tissue, thereby confirming effectiveness of the chosen experimental model and the pathological course.

FIG. 3. MMP-13 expression on specimen of joint of rat subjected to destabilisation of medial meniscus. Presence of MMP-13 expressed according to colour intensity (greyscale).

As expected, specimens obtained from untreated animals showed a marked presence of MMP-13 in the cartilaginous tissue, thereby confirming effectiveness of the chosen experimental model and the pathological course.

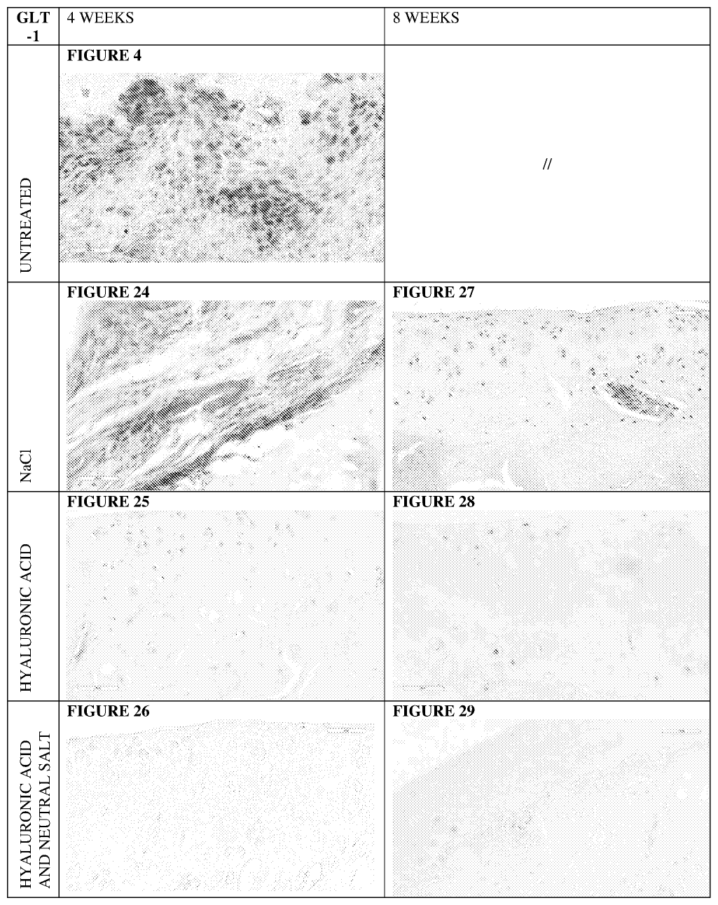

FIG. 4. GLT-1 expression on specimen of joint of rat subjected to destabilisation of medial meniscus. Presence of GLT-1 expressed according to colour intensity (greyscale).

As expected, specimens obtained from untreated animals showed a marked presence of GLT-1 in the cartilaginous tissue, thereby confirming effectiveness of the chosen experimental model and the pathological course.

FIG. 5. GLT-3 expression on specimen of joint of rat subjected to destabilisation of medial meniscus. Presence of GLT-3 expressed according to colour intensity (greyscale).

As expected, specimens obtained from untreated animals showed a marked presence of GLT-3 in the cartilaginous tissue, thereby confirming effectiveness of the chosen experimental model and the pathological course.

FIGS. 6-8 and 9-11 refer to specimens treated and then stained with a specific probe for Collagen II. The images were acquired respectively at 4 weeks and 8 weeks from start of treatment:

FIG. 6. Collagen II expression on specimen of joint of rat subjected to destabilisation of medial meniscus, treated with intra-articular infiltration of saline solution. Presence of Collagen II expressed according to colour intensity (greyscale).

As can be seen in FIG. 6, after 4 weeks, in the articular tissue of rats treated with saline solution, Collagen II was not particularly expressed. This result is superimposable on that obtained in the absence of treatment and indicates that intra-articular infiltration with saline solution does not lead to recovery of the structure and mechanical characteristics of healthy cartilaginous tissue.

FIG. 7. Collagen II expression on specimen of joint of rat subjected to destabilisation of medial meniscus, treated with intra-articular infiltration of hyaluronic acid. Presence of Collagen II expressed according to colour intensity (greyscale).

As can be seen in FIG. 7, after 4 weeks, in the articular tissue of rat subjected to destabilisation of medial meniscus, treated with hyaluronic acid, Collagen II expression improved with respect to data obtained in absence of treatment or with intra-articular infiltration of saline solution. This result indicates that treatment with hyaluronic acid results in improvement in the structure and mechanical characteristics of cartilaginous tissue in pathological conditions.

FIG. 8. Collagen II expression on specimen of joint of rat subjected to destabilisation of medial meniscus, treated with intra-articular infiltration of hyaluronic acid and neutral salt (Example 21). Presence of Collagen II expressed according to colour intensity (greyscale).

As can be seen in FIG. 8, after 4 weeks, in the articular tissue of rat subjected to destabilisation of medial meniscus, treated with a solution of hyaluronic acid and neutral salt, Collagen II expression improved particularly with respect to data obtained in absence of treatment or with intra-articular infiltration of saline solution. This result indicates that treatment with the composition envisaged in the invention results in a clear improvement in the structure and mechanical characteristics of cartilaginous tissue in pathological conditions.

FIG. 9. Collagen II expression on specimen of joint of rat subjected to destabilisation of medial meniscus, treated with intra-articular infiltration of saline solution. Presence of Collagen II expressed according to colour intensity (greyscale).

As can be seen in FIG. 9, in the articular tissue of rat subjected to destabilisation of medial meniscus, treated with saline solution, Collagen II was not particularly expressed after 8 weeks either. This result confirms the data observed at 4 weeks and is superimposable on that obtained in absence of treatment. Intra-articular infiltrated saline solution was not effective in promoting formation of Collagen II and in promoting, therefore, an improvement in the structure and characteristics of pathological cartilage.

FIG. 10. Collagen II expression on specimen of joint of rat subjected to destabilisation of medial meniscus, treated with intra-articular infiltration of hyaluronic acid. Presence of Collagen II expressed according to colour intensity (greyscale).

As can be seen in FIG. 10, in the articular tissue of rat subjected to destabilisation of medial meniscus, treated with hyaluronic acid, Collagen II expression improved with respect to data obtained in absence of treatment or with intra-articular infiltration of saline solution, also after 8 weeks. This result indicates that treatment with hyaluronic acid leads to improvement in the structure and mechanical characteristics of cartilaginous tissue in pathological conditions.

FIG. 11. Collagen II expression on specimen of joint of rat subjected to destabilisation of medial meniscus, treated with intra-articular infiltration of hyaluronic acid and neutral salt (Example 21). Presence of Collagen II expressed according to colour intensity (greyscale).

As can be seen in FIG. 11, in the articular tissue of rat subjected to destabilisation of medial meniscus, treated with solution of hyaluronic acid and neutral salt, Collagen II expression improved particularly with respect to data obtained in absence of treatment or with intra-articular infiltration of saline solution also after 8 weeks. This result indicates that treatment with the composition envisaged in the invention results in a clear improvement in the structure and mechanical characteristics of cartilaginous tissue in pathological conditions.

FIGS. 12-14 and 15-17 refer to specimens treated and then stained with a specific probe for MMP-3. The images were acquired respectively at 4 weeks and 8 weeks from start of treatment:

FIG. 12. MMP-3 expression on specimen of joint of rat subjected to destabilisation of medial meniscus, treated with intra-articular infiltration of saline solution. Presence of MMP-3 expressed according to colour intensity (greyscale).

As can be seen in FIG. 12, after 4 weeks, in the articular tissue of rat subjected to destabilisation of medial meniscus, treated with saline solution, MMP-3 was particularly expressed. This result is superimposable on that obtained in the absence of treatment and indicates that intra-articular infiltration with saline solution does not improve inflammatory response in pathological conditions.

FIG. 13. MMP-3 expression on specimen of joint of rat subjected to destabilisation of medial meniscus, treated with intra-articular infiltration of hyaluronic acid. Presence of MMP-3 expressed according to colour intensity (greyscale).

As can be seen in FIG. 13, after 4 weeks, in the articular tissue of rat subjected to destabilisation of medial meniscus, treated with hyaluronic acid, MMP-3 expression was lower than that observed in absence of treatment or after treatment with saline solution. This result indicates that intra-articular infiltration with hyaluronic acid improves inflammatory response in pathological conditions.

FIG. 14. MMP-3 expression on specimen of joint of rat subjected to destabilisation of medial meniscus, treated with intra-articular infiltration of hyaluronic acid and neutral salt (Example 21). Presence of MMP-3 expressed according to colour intensity (greyscale).

As can be seen in FIG. 14, after 4 weeks, in the articular tissue of rat subjected to destabilisation of medial meniscus, treated with hyaluronic acid and neutral salt, MMP-3 expression was practically absent with respect to that observed in absence of treatment or after treatment with saline solution or with hyaluronic acid. This result indicates that intra-articular infiltration with the composition envisaged in the invention significantly improves inflammatory response in pathological conditions, thereby offering better performances also with respect to infiltration solely with hyaluronic acid.

FIG. 15. MMP-3 expression on specimen of joint of rat subjected to destabilisation of medial meniscus, treated with intra-articular infiltration of saline solution. Presence of MMP-3 expressed according to colour intensity (greyscale).

As can be seen in FIG. 15, in the articular tissue of rat subjected to destabilisation of medial meniscus, treated with saline solution, MMP-3 was particularly expressed also after 8 weeks. This result is superimposable on that obtained at 4 weeks in the absence of treatment and indicates that intra-articular infiltration with saline solution does not improve inflammatory response in pathological conditions.

FIG. 16. MMP-3 expression on specimen of joint of rat subjected to destabilisation of medial meniscus, treated with intra-articular infiltration of hyaluronic acid. Presence of MMP-3 expressed according to colour intensity (greyscale).

As can be seen in FIG. 16, in the articular tissue of rat subjected to destabilisation of medial meniscus, treated with hyaluronic acid, MMP-3 expression was lower than that observed in absence of treatment or after treatment with saline solution also at 8 weeks. This result indicates that intra-articular infiltration with hyaluronic acid improves inflammatory response in pathological conditions but does not stop the inflammatory focus.

FIG. 17. MMP-3 expression on specimen of joint of rat subjected to destabilisation of medial meniscus, treated with intra-articular infiltration of hyaluronic acid and neutral salt (Example 21). Presence of MMP-3 expressed according to colour intensity (greyscale).

As can be seen in FIG. 17, in the articular tissue of rat subjected to destabilisation of medial meniscus, treated with hyaluronic acid and neutral salt, MMP-3 expression was practically absent with respect to that observed in absence of treatment or after treatment with saline solution or with hyaluronic acid also at 8 weeks. This result indicates that intra-articular infiltration with the composition envisaged in the invention significantly improves inflammatory response in pathological conditions, thereby offering better performances also with respect to infiltration solely with hyaluronic acid and above all calms inflammatory focus for a prolonged period of time.

FIGS. 18-20 and 21-23 refer to specimens treated and then stained with a specific probe for MMP-13. The images were acquired respectively at 4 weeks and 8 weeks from start of treatment:

FIG. 18. MMP-13 expression on specimen of joint of rat subjected to destabilisation of medial meniscus, treated with intra-articular infiltration of saline solution. Presence of MMP-13 expressed according to colour intensity (greyscale).

As can be seen in FIG. 18, after 4 weeks, in the articular tissue of rat subjected to destabilisation of medial meniscus treated with saline solution, MMP-13 is particularly expressed. This result is superimposable on that obtained in the absence of treatment and indicates that intra-articular infiltration with saline solution does not improve inflammatory response in pathological conditions.

FIG. 19. MMP-13 expression on specimen of joint of rat subjected to destabilisation of medial meniscus, treated with intra-articular infiltration of hyaluronic acid. Presence of MMP-13 expressed according to colour intensity (greyscale).

As can be seen in FIG. 19, after 4 weeks, in the articular tissue of rat subjected to destabilisation of medial meniscus treated with hyaluronic acid, MMP-13 expression was not particularly lower than that observed in absence of treatment or after treatment with saline solution. This result indicates that intra-articular infiltration with hyaluronic acid does not improve inflammatory response in pathological conditions.

FIG. 20. MMP-13 expression on specimen of joint of rat subjected to destabilisation of medial meniscus, treated with intra-articular infiltration of hyaluronic acid and neutral salt (Example 21). Presence of MMP-13 expressed according to colour intensity (greyscale).

As can be seen in FIG. 20, after 4 weeks, in the articular tissue of rat subjected to destabilisation of medial meniscus, treated with hyaluronic acid and neutral salt, MMP-13 expression was much lower than that observed in absence of treatment or after treatment with saline solution or solely with hyaluronic acid. This result indicates that intra-articular infiltration with the composition envisaged in the invention improves inflammatory response in pathological conditions, thereby offering better performances also with respect to infiltration solely with hyaluronic acid.

FIG. 21. MMP-13 expression on specimen of joint of rat subjected to destabilisation of medial meniscus, treated with intra-articular infiltration of saline solution. Presence of MMP-13 expressed according to colour intensity (greyscale).

As can be seen in FIG. 21, in the articular tissue of rat subjected to destabilisation of medial meniscus treated with saline solution, MMP-13 was particularly expressed also after 8 weeks. This result is worse than that obtained at 4 weeks in the absence of treatment and indicates that intra-articular infiltration with saline solution does not improve inflammatory response in pathological conditions.

FIG. 22. MMP-13 expression on specimen of joint of rat subjected to destabilisation of medial meniscus, treated with intra-articular infiltration of hyaluronic acid. Presence of MMP-13 expressed according to colour intensity (greyscale).

As can be seen in FIG. 22, in the articular tissue of rat subjected to destabilisation of medial meniscus treated with hyaluronic acid, MMP-13 expression was lower than that observed in absence of treatment or after treatment with saline solution at 8 weeks and with hyaluronic acid at 4 weeks. This result indicates that intra-articular infiltration with hyaluronic acid improves inflammatory response in pathological conditions, bringing greater benefits in the chronic phase of the disease than in the acute phase thereof.

FIG. 23. MMP-13 expression on specimen of joint of rat subjected to destabilisation of medial meniscus, treated with intra-articular infiltration of hyaluronic acid and neutral salt (Example 21). Presence of MMP-13 expressed according to colour intensity (greyscale).

As can be seen in FIG. 23, in the articular tissue of rats suffering from osteoarthritis, treated with hyaluronic acid and neutral salt, MMP-13 expression was much lower than that observed in absence of treatment or after treatment with saline solution or solely with hyaluronic acid also at 8 weeks. This result indicates that intra-articular infiltration with hyaluronic acid and neutral salt significantly improves inflammatory response in pathological conditions, thereby offering better performances also with respect to infiltration solely with hyaluronic acid and above all calms inflammatory focus for a prolonged period of time, acting in both the acute phase and the chronic phase of the disease.

FIGS. 24-26 and 27-29 refer to specimens treated and then stained with a specific probe for GLT-1. The images were acquired respectively at 4 weeks and 8 weeks from start of treatment:

FIG. 24. MMP-1 expression on specimen of joint of rat subjected to destabilisation of medial meniscus, treated with intra-articular infiltration of saline solution. Presence of GLT-1 expressed according to colour intensity (greyscale).

As can be seen in FIG. 24, after 4 weeks, in the articular tissue of rat subjected to destabilisation of medial meniscus treated with saline solution, GLT-1 was particularly expressed. This result is superimposable on that obtained in the absence of treatment and indicates that intra-articular infiltration with saline solution does not improve expression of one of the key receptors involved in the genesis of the inflammatory cascade.

FIG. 25. GLT-1 expression on specimen of joint of rat subjected to destabilisation of medial meniscus, treated with intra-articular infiltration of hyaluronic acid. Presence of GLT-1 expressed according to colour intensity (greyscale).

As can be seen in FIG. 25, after 4 weeks, in the articular tissue of rat subjected to destabilisation of medial meniscus treated with hyaluronic acid, GLT-1 was not particularly expressed. This result indicates that intra-articular infiltration with hyaluronic acid improves expression of one of the key receptors involved in the genesis of the inflammatory cascade.

FIG. 26. GLT-1 expression on specimen of joint of rat subjected to destabilisation of medial meniscus, treated with intra-articular infiltration of hyaluronic acid and neutral salt (Example 21). Presence of MMP-1 expressed according to colour intensity (greyscale).

As can be seen in FIG. 26, after 4 weeks, in the articular tissue of rat subjected to destabilisation of medial meniscus treated with hyaluronic acid and neutral salt, GLT-1 expression was practically absent with respect to that observed in absence of treatment or after treatment with saline solution. This result indicates that intra-articular infiltration with the composition envisaged in the invention significantly improves expression of one of the key receptors involved in the genesis of the inflammatory cascade.

FIG. 27. GLT-1 expression on specimen of joint of rat subjected to destabilisation of medial meniscus, treated with intra-articular infiltration of saline solution. Presence of GLT-1 expressed according to colour intensity (greyscale).

As can be seen in FIG. 27, in the articular tissue of rat subjected to destabilisation of medial meniscus treated with saline solution, GLT-1 expression was lower after 8 weeks. This result is an improvement on that obtained at 4 weeks and in the absence of treatment and indicates that intra-articular infiltration with saline solution does not prevent improvements in expression of one of the key receptors involved in the genesis of the inflammatory cascade in the chronic phase of the disease.

FIG. 28. GLT-1 expression on specimen of joint of rat subjected to destabilisation of medial meniscus, treated with intra-articular infiltration of hyaluronic acid. Presence of GLT-1 expressed according to colour intensity (greyscale).

As can be seen in the figure, in the articular tissue of rat subjected to destabilisation of medial meniscus treated with hyaluronic acid, GLT-1 expression was practically absent at 8 weeks. This result indicates that intra-articular infiltration with hyaluronic acid improves expression of one of the key receptors involved in the genesis of the inflammatory cascade and keeps it improved for a prolonged period.

FIG. 29. GLT-1 expression on specimen of joint of rat subjected to destabilisation of medial meniscus, treated with intra-articular infiltration of hyaluronic acid and neutral salt (Example 21). Presence of GLT-1 expressed according to colour intensity (greyscale).

As can be seen in FIG. 29, in the articular tissue of rat subjected to destabilisation of medial meniscus treated with hyaluronic acid and neutral salt, GLT-1 expression was practically absent. This result indicates that intra-articular infiltration with the composition envisaged in the invention improves expression of one of the key receptors involved in the genesis of the inflammatory cascade and efficaciously maintains performance for a prolonged period.

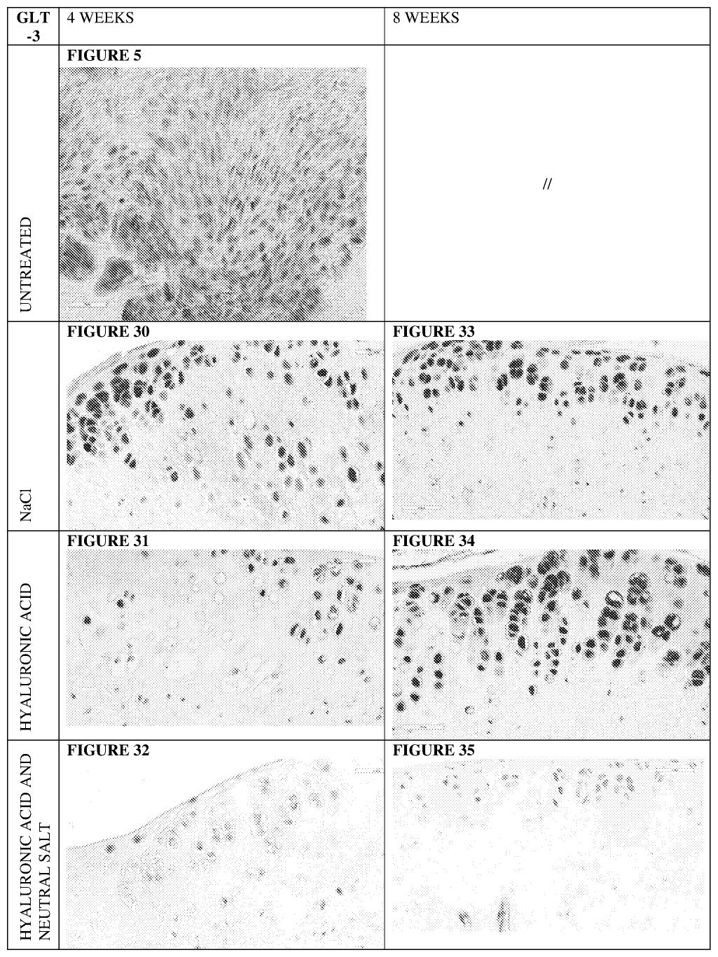

FIGS. 30-32 and 33-35 refer to specimens treated and then stained with a specific probe for GLT-3. The images were acquired respectively at 4 weeks and 8 weeks from start of treatment:

FIG. 30. GLT-3 expression on specimen of joint of rat subjected to destabilisation of medial meniscus, treated with intra-articular infiltration of saline solution. Presence of MMP-3 expressed according to colour intensity (greyscale).

As can be seen in FIG. 30, after 4 weeks, in the articular tissue of rat subjected to destabilisation of medial meniscus treated with saline solution, GLT-3 was particularly expressed. This result is superimposable on that obtained in the absence of treatment and indicates that intra-articular infiltration with saline solution does not improve expression of one of the key receptors involved in the genesis of the inflammatory cascade.

FIG. 31. GLT-3 expression on specimen of joint of rat subjected to destabilisation of medial meniscus, treated with intra-articular infiltration of hyaluronic acid. Presence of MMP-3 expressed according to colour intensity (greyscale).

As can be seen in FIG. 31, after 4 weeks, in the articular tissue of rat subjected to destabilisation of medial meniscus treated with hyaluronic acid, GLT-3 was noticeably expressed. This result indicates that intra-articular infiltration with hyaluronic acid improves, solely partly, expression of one of the key receptors involved in the genesis of the inflammatory cascade.

FIG. 32. GLT-3 expression on specimen of joint of rat subjected to destabilisation of medial meniscus, treated with intra-articular infiltration of hyaluronic acid and neutral salt (Example 21). Presence of GLT-3 expressed according to colour intensity (greyscale).

As can be seen in FIG. 32, after 4 weeks, in the articular tissue of rat subjected to destabilisation of medial meniscus treated with hyaluronic acid and neutral salt, GLT-3 expression was practically absent with respect to that observed in absence of treatment or after treatment with saline solution. This result indicates that intra-articular infiltration with the composition envisaged in the invention improves expression of one of the key receptors involved in the genesis of the inflammatory cascade.

FIG. 33. GLT-3 expression on specimen of joint of rat subjected to destabilisation of medial meniscus, treated with intra-articular infiltration of saline solution. Presence of GLT-3 expressed according to colour intensity (greyscale).

As can be seen in FIG. 33, in the articular tissue of rat subjected to destabilisation of medial meniscus treated with saline solution, GLT-3 expression was identical after 8 weeks. This result is not an improvement on that obtained at 4 weeks and in the absence of treatment and indicates that intra-articular infiltration with saline solution does not produce any improvements in expression of one of the key receptors involved in the genesis of the inflammatory cascade in the chronic phase of the disease.

FIG. 34. GLT-3 expression on specimen of joint of rat subjected to destabilisation of medial meniscus, treated with intra-articular infiltration of hyaluronic acid. Presence of GLT-3 expressed according to colour intensity (greyscale).

As can be seen in FIG. 34, in the articular tissue of rat subjected to destabilisation of medial meniscus treated with hyaluronic acid, GLT-3 expression was clearly worse at 8 weeks. This result clearly indicates that intra-articular infiltration with hyaluronic acid does not improve expression of one of the key receptors involved in the genesis of the inflammatory cascade and keep it improved for a prolonged period.

FIG. 35. GLT-3 expression on specimen of joint of rat subjected to destabilisation of medial meniscus, treated with intra-articular infiltration of hyaluronic acid and neutral salt (Example 21). Presence of GLT-3 expressed according to colour intensity (greyscale).

As can be seen in FIG. 35, in the articular tissue of rat subjected to destabilisation of medial meniscus, treated with hyaluronic acid and neutral salt, GLT-3 expression was particularly low. This result indicates that intra-articular infiltration with the composition envisaged in the invention improves expression of one of the key receptors involved in the genesis of the inflammatory cascade and efficaciously maintains performance for a prolonged period.

The association of the bioactive substance and neutral salt surprisingly provides not only an improvement in performance but also prolongs the positive effects of the treatment. Indeed, the association envisaged in the invention not only improves production of Collagen II at 4 weeks (FIGS. 7 and 8), but also and above all offers a continued effect and further improvement at 8 weeks (FIG. 11). Likewise, also for reduced expression of MMP13, in the case of the association envisaged in the invention, there is progressive improvement from Weeks 4 to 8 (FIGS. 20 and 23) which is greater than with solely the active substance (FIGS. 19 and 22). Going beyond all expectations, only the association of the bioactive substance and neutral salt maintains the reduced expression of GLT-3 (FIGS. 32 and 35) over time, while the active substance alone shows, on the contrary, an increased production in the marker from Weeks 4 to 8 (FIGS. 31 and 34).

1. A method for treatment of joint tissues affected by arthropathy, said method comprising the step of administering a therapeutically effective amount of a pharmaceutical composition to patients in need thereof,
wherein said pharmaceutical composition reduces the expression of the GLT3 receptor and the MMP13 enzyme, and increases the production of collagen II, and wherein said pharmaceutical composition comprises:
at least a bioactive substance selected from collagen, fibrinogen, fibrin, alginic acid, sodium alginate, potassium alginate, magnesium alginate, hyaluronic acid, sodium hyaluronate, potassium hyaluronate, iron hyaluronate, calcium hyaluronate, magnesium hyaluronate, zinc hyaluronate, hyaluronic acid derivate, cellulose, chondroitin sulfate, dermatan sulfate, keratan sulfate, heparin, heparan sulfate, laminin, fibronectin, elastin, polylactic acid, polyglycolic acid, poly(lactic-co-glycolic acid), polycaprolactone, gelatin, albumin, poly(glycolide-co-caprolactone), poly(glycolide-co-trimethylene carbonate), hydroxyapatite, tricalcium phosphate, dicalcium phosphate, demineralized bone matrix, and mixtures thereof, and
at least one neutral salt consisting of a polyamino-saccharide cation and an anion, wherein the polyamino-saccharide cation consists of the following three repeating units: 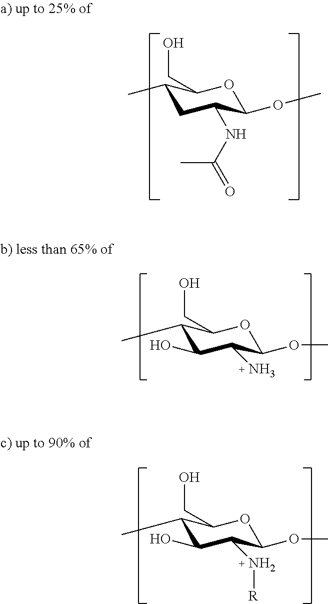
wherein R an aldose or ketose moiety,
and wherein the anion is monovalent, bivalent or trivalent.

2. The method of claim 1, wherein said pharmaceutical composition comprises one bioactive substance and one neutral salt.

3. The method of claim 1, wherein the pharmaceutical composition further reduces the expression of the GLT1 receptor.

4. The method of claim 1, wherein the pharmaceutical composition of the invention further reduces the expression of the MMP3 enzyme.

5. The method of claim 1, wherein said pharmaceutical composition has a pH of 6-8.

6. The method of claim 1, wherein said pharmaceutical composition further comprises a buffer, preferably a saline phosphate buffer.

7. The method of claim 1, wherein said pharmaceutical composition is in the form of an aqueous solution for injection.

8. The method of claim 1, wherein, in said at least one neutral salt, R is a moiety of formula (1): 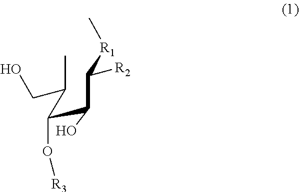
wherein R₁ is —CH₂— or —CO—,
R₂ is —OH, or —NHCOCH₃,
R₃ is H, monosaccharide, disaccharide, or oligosaccharide,
or R is a moiety of formula (2): 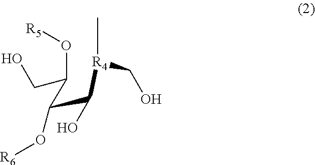
R₄ is —CH—,
R5 and R6 are, independently of each other, H, monosaccharide, disaccharide, or oligosaccharide.

9. The method of claim 8, wherein, in said at least one neutral salt, R₃, R₅ e R₆ are, independently of one another, H, glucose, galactose, arabinose, xylose, mannose, lactose, trealose, gentiobiose, cellobiose, cellotriose, maltose, maltotriose, chitobiose, chitotriose, mannobiose, melibiose, fructose, N-acetyl glucosamine, N-acetylgalactosamine, or a combination thereof.

10. The method of claim 1, wherein, in said at least one neutral salt, R is a lactose or galactose moiety.

11. The method of claim 1, wherein, in the polyamino-saccharide cation of the at least one neutral salt, the repeating unit a) is present in a percentage of 5% to 20%

12. The method of claim 1, wherein, in the polyamino-saccharide cation of the at least one neutral salt, the repeating unit b) is present in a percentage of 5% to 45%.

13. The method of claim 1, wherein, in the polyamino-saccharide cation of the at least one neutral salt, the repeating unit c) is present in a percentage of 45% to 90%.

14. The method of claim 1, wherein, in said at least one neutral salt, the anion is chloride, bromide, fluoride, iodide, acetate, trifluoroacetate, carbonate, bicarbonate, sulfate, bisulfate, C1-C10 alkyl sulfate, C1-C6 alkylsulfonate, C6-C10 arylsulfonate, nitrate, hydrogen phosphate, dihydrogen phosphate, orthophosphate, oxalate, fumarate, ascorbate, citrate, gluconate, lactate, formate, tartrate, succinate, mandelate, p-toluenesulfonate, carboxylate, saccharate, benzoate, or a mixture thereof.

15. The method of claim 1, wherein the weight average molecular weight of the at least one neutral salt is up to 2500 kDa, or the number average molecular weight of the at least one neutral salt is up to 2000 kDa.

16. The method of claim 1, wherein said at least one neutral salt and said at least one bioactive substance are in a ratio of 10:1 to 1:50.

17. The method of claim 16, wherein said at least one neutral salt and said at least one bioactive substance are in a ratio of 5:1 to 1:5.

18. The method of claim 17, wherein said bioactive substance is selected from hyaluronic acid, sodium hyaluronate, potassium hyaluronate, iron hyaluronate, calcium hyaluronate, magnesium hyaluronate, zinc hyaluronate, hyaluronic acid derivate, and mixtures thereof.
